(19) United States
(12) Patent Application Publication
(10) Pub. No.: US 2003/0036195 A1
(43) Pub. Date: Feb. 20, 2003

(54) Generation of differentiated tissue from nuclear transfer embryonic stem cells and methods of use

(76) Inventors: Lorenz Studer, New York, NY (US); Viviane Tabar, New York, NY (US); Peter Mombaerts, New York, NY (US); Teruhiko Wakayama, Chuou-ku (JP); Anthony Perry, Chuo-ku (JP)

Correspondence Address: KLAUBER & JACKSON, 411 HACKENSACK AVENUE, HACKENSACK, NJ, 07601

(21) Appl. No.: 10/127,740

(22) Filed: Apr. 22, 2002

Related U.S. Application Data
(60) Provisional application No. 60/285,654, filed on Apr. 20, 2001.

(51) Int. Cl. C12N 5/08

(52) U.S. Cl. 435/368

(57) ABSTRACT

The present invention provides methods of preparing mammalian cells and tissues for therapeutic and diagnostic purposes that are derived from ntES cells. The present invention further provides the mammalian cells and tissues themselves. In addition, methods of using the mammalian cells and tissues as a therapeutic agent or as a diagnostic are provided.

### GOVERNMENTAL SUPPORT

The research leading to the present invention was supported, at least in part, by the National Institute of Cancer CORE Grant No. 08748. Accordingly, the U.S. Government may have certain rights in the invention.

### FIELD OF THE INVENTION

The present invention relates to the preparation of mammalian cells and tissues for therapeutic and diagnostic purposes. These mammalian cells and tissues are generated from embryonic cell lines generated by the transfer of the nucleus of an adult somatic cell to an enucleated oocyte (i.e., nuclear transfer embryonic stem cells).

### BACKGROUND OF THE INVENTION

In nature, all of the cells and cell types of an individual adult mammal are derived from a single undifferentiated cell, a fertilized oocyte, i.e., the zygote. The zygote is also the precursor of certain non-embryonic cells, such as the cells that make up the placenta. At the other extreme, most adult cells are fully differentiated and normally cannot be converted into another cell type. One particular exception is the adult stem cell. Adult stem cells retain the ability to differentiate into other cell types, though even adult stem cells are generally limited to forming cells of a single tissue type. Thus, hematopoietic stem cells are capable of differentiating into any cell type of the blood, whereas brain stem cells can differentiate into the different cell types of the brain. In contrast to adult stem cells, embryonic stem cells (ES) are not tissue-limited, but are pluripotent and can differentiate into multiple cell types, though unlike the totipotent zygote, ES cells are limited to forming cells derived from the embryo.

ES cells have generated a great deal of interest in recent years since a tissue, organ or even an individual animal can, at least in theory, be grown de novo from a single ES cell. Thus, ES cells obtained from animals having desirable properties could be particularly valuable in animal husbandry. In addition, such technology may even find a use in forming herds of livestock free of deleterious prions. Similarly, tissues derived from ES cells could be used in tissue and organ transplants. Moreover, ES cells could have great therapeutic value in treating diseases in which key cells are depleted, such as in insulin-dependent diabetes and in Parkinson's disease. Currently, however, obtaining ES cells to carry out these procedures has been problematic.

One source of ES cells is of course embryonic/fetal tissue. ES cell lines also have been constructed that are have been derived from cells of the developing blastocyst, an early stage in embryonic development that consists of a hollow ball of embryonic cells. Such ES cell lines can proliferate extensively and be induced to differentiate ultimately into multiple adult cell types. For obvious ethical considerations however, an alternative source of human embryonic stem cells is extremely desirable.

In a related technology, individual mammals have been generated through nuclear transfer cloning, with Dolly the sheep being the most famous result. Dolly was produced through the electrofusion of a cultured sheep mammary gland cell with enucleated sheep oocyte and subsequent transplantation into a surrogate mother [Wilmut et al., Nature 385, 810-813 (1997)]. In an alternative procedure, the nucleus of an adult somatic mouse cell was directly inserted into an enucleated mouse oocyte, which after subsequent transplantation into a surrogate mother, resulted in a mouse that had the identical nuclear genome of the somatic cell [WO 99/37143 (1999)].

More recently, it has been suggested that such “nuclear transfer” methodology could be used to generate an alternative source of ES cells, namely nuclear transfer embryonic stem cells (ntES cells) [Aldhous, Nature 410, 622-625 (2001)]. Besides overcoming the potential ethical issues mentioned above, this source of pluripotent cells also can provide a perfect immunological match for a cell/tissue transplant since the cell/tissue can be generated with the genetic make-up of a somatic cell obtained from the ultimate recipient. Unfortunately however, attempts to construct an ntES cell capable of such use have been unsuccessful [Munsie et al., Curr.Biol. 10, 989-992 (2000); Kawase et al., Genesis 28,156-163 (2000)]. Indeed, heretofore, no ntES cell has been obtained which contributes to the germ line, and the ability to contribute to the germ line is considered a defining characteristic of ES cells. Furthermore, heretofore, the process of constructing ntES cells has been, at best disappointingly inefficient, and the progress for increasing the efficiency, has currently been described as being “stalled” [Aldhous, Nature 410, 622-625 (2001)].

Therefore, there is a need to provide a source of ntES cells that is capable of contributing to the germ line. In addition, there is a need to devise methods of generating differentiated cells from nuclear transfer ES cells that can be used in the treatment of human diseases. More particularly, there is a need to devise methods of generating neuronal cells from nuclear transfer ES cells that can be used in the treatment of Parkinson's disease. Furthermore, there is a need to provide the differentiated cells and tissues obtained by these methods.

The citation of any reference herein should not be construed as an admission that such reference is available as “Prior Art” to the instant application.

### SUMMARY OF THE INVENTION

The present invention provides a novel source of differentiated cells and tissues. These cells and tissues are generated from nuclear transfer embryonic stem (ntES) cells. By employing the ntES cells of the present invention, the present invention allows the production of de novo cells, tissues, and organs that comprise the identical genetic material of a live animal. Such cells, tissues, and organs can thus be specifically tailored for the animal recipient.

The present invention therefore provides methods of generating differentiated cells from ntES cells. One such embodiment comprises generating an embryoid body (EB) from an ntES cell. The embryoid body is then treated with growth factors and mitogens to begin differentiation. Finally the mitogen is withdrawn to complete the process. In this manner a differentiated cell is formed. The differentiated cell then can be used to generate a tissue or organ. The cells, tissues, and organs generated are also part of the present invention.

In one particular aspect of the present invention methods of generating a neuronal cell are provided. One such method comprises culturing a nuclear transfer embryonic stem (ntES) cell in a first container, whereby an embryoid body (EB) is formed. The resulting embryoid body is removed from the first container, resuspended in appropriate medium and then placed in a second container. Appropriate medium preferably contains “knock out” DMEM (Dulbecco's modified Eagle medium) or equivalent basal medium supplemented with 10-20% ES qualified serum or serum replacement. In addition supplements are required such as beta-mercaptoethanol, non-essential amino acids MEM and glutamine (which is particularly preferred for this specific application). Nucleosides on the other hand may be omitted. In a prefered embodiment, the medium is ES cell medium (see Example 2, below).

The ES cell medium is then removed from the second container and it is replaced with serum free media supplemented with an attachment factor. In a particular embodiment, the attachment factor is laminin. In another embodiment, the attachment factor is collagen. In still another embodiment, the attachment factor is polylysine. In yet another embodiment, the attachment factor is entactin-collagen-laminin (ECL. In a preferred embodiment, the attachment factor is fibronectin.

The embryoid body is then allowed to grow for 9 or more days (preferably 9 to 16 days) at which time the embryoid body expresses the neural stem cell marker nectin. The embryoid body expressing nectin is then removed from the second container and placed in a third container coated with polyomithine/lamininin. The medium is then supplemented with a mitogen, laminin, sonic hedgehog and FGF8. Finally the mitogen is withdrawn from the medium (e.g., the media is replaced with media that does not contain the mitogen) and a differentiated neuronal cell is formed.

In one embodiment the method is specific for generating a dopaminergic neuron. In a particular embodiment of this type, the mitogen is bFGF. In a preferred embodiment of this type, ascorbic acid is added along with the mitogen, laminin, sonic hedgehog and FGF8 when the embryoid body is placed in the container coated with polyomithine/lamininin. In still another embodiment, one or more of the following factors: retinoic acid, a retinoic acid derivative such as 9-cis retinoic acid, 13-cis-retionic acid and/or all-trans retinoic acid, BDNF, NT4, a bone morphogenetic protein such as BMP2, BMP4, and/or BMP7, GDNF, neurturin, artemin, dbbcAMP, pax2, pax5, pax8, Nurr1, ptx3, and 1mx1b are added to the medium with the mitogen, laminin, sonic hedgehog and FGF8 when the embryoid body is placed in the container coated with polyornithine/lamininin and/or during the step immediately preceding it.

In another embodiment the method is specific for generating a serotonergic neuron. In a particular embodiment of this type, the mitogen is bFGF.

In still another embodiment the method is specific for generating an astrocyte. In one such method, following the step of placing the embryoid body in the third container, i.e., coated with polyomithine/lamininin, and adding the mitogen, laminin, sonic hedgehog and FGF8 to the medium, but prior to the step in which the mitogen is withdrawn, the embryoid body is removed from the third container and then proliferated on a fourth container with a mitogen selected from the group consisting of bFGF, EGF, and PDGF.

In yet another embodiment, the method is specific for generating an oligodendrocyte, In one such method, following the step of placing the embryoid body in the third container, i.e., coated with polyornithine/lamininin, and adding the mitogen, laminin, sonic hedgehog and FGF8 to the medium the embryoid body is removed from the third container and then proliferated in a fourth container with bFGF plus EGF and bFGF plus CNTF (of LIF). The final step is then performed in media in which the bFGF plus EGF and the bFGF plus CNTF (of LIF) are withdrawn.

In still another embodiment, the method is specific for generating a GABA neuron. In one such method, when the embryoid body is placed in the container coated with polyomithine/lamininin, the mitogen and laminin, but not the sonic hedgehog and FGF8 are added to the medium and the final step of withdrawal of the mitogen is performed in the presence of dbcAMP and BDNF or NT4.

In a related aspect of the present invention a neuronal cell produced from an ntES cell is provided. In a preferred embodiment the neuronal cell is produced ex vivo. In one such embodiment the neuronal cell is a serotonergic neuron. In still another embodiment the neuronal cell is an astrocyte. In yet another embodiment the neuronal cell is a GABA neuron. In still another embodiment the neuronal cell is an oligodendrocyte. In preferred embodiment, the neuronal cell is a dopaminergic neuron.

Accordingly, it is a principal object of the present invention to provide a neuronal cell that has been produced from a nuclear transfer embryonic stem cell.

It is a further object of the present invention to provide a dopaminergic neuron derived from a nuclear transfer embryonic stem cell.

It is a further object of the present invention to provide an efficient means of generating ntES cells.

It is a further object of the present invention to provide a method of generating a differentiated cell from an ntES cell.

It is a further object of the present invention to provide a treatment of Parkinson's disease using a dopaminergic neuron derived from a nuclear transfer embryonic stem cell.

These and other aspects of the present invention will be better appreciated by reference to the following drawings and Detailed Description.

### BRIEF DESCRIPTION OF THE DRAWINGS

FIGS. 1A-1E show the dopaminergic and serotonergic differentiation of ntES cells in vitro. Embryoid bodies were plated under conditions favoring CNS selection followed by dopaminergic induction. Images shown are of C15. FIG. 1A shows the colocalization of tyrosine-hydroxylase (TH, green) and β-III tubulin (red). FIG. 1B shows the presence of serotonergic (Ser, green) and TH (red) neurons. Scale bar=100 μm. FIG. 1C shows the yield of TH⁺ neurons varied among the cell lines tested, with>50% of total cell number in C15 cells. Other commonly used ES lines (E14, AB2.2) generated a percentage of TH⁺ cells falling within the range shown by the ntES cells. C4, C15, C16, CN1, CN2, CT1, CT2 represent ntES, and AB2.2 and E14 ES cell lines. FIG. 1D is a representative chromatogram showing elution and electrochemical detection of dopamine (DA) and serotonin (Ser) from conditioned medium by reverse phase HPLC. FIG 1E shows the quantification of dopamine and serotonin release. Neurotransmitter concentration was determined in conditioned medium (CM; 24 hours after last medium change), basal condition (15 minutes in buffer solution) and upon evoked release (KCl; 15 minutes in 56 mM KCl buffer). Serotonin release was low under basal and evoked conditions, probably reflecting a lower number of serotonergic neurons.

FIGS. 2A-2D demonstrates totipotency of ntES cells in vivo. FIG. 2A demonstrates the contribution of C57BL/6^{nu/nu}-nudentES cells (line CN1) to chimeric offspring following injection into ICR×ICR fertilization-derived blastocysts in offspring 14 days after birth in which the dark coat color derives from the ntES cell contribution. In FIG. 2B the male indicated with an asterisk in FIG. 2A was crossed at 8 weeks with a white (ICR) female, producing a litter containing three dark offspring, confirming the contribution of C57BL/6^{nu} to the germ line. Asterisks in FIGS. 2A and 2B indicate the same male. Cloning using ntES cells as nucleus donors shown in FIG. 2C, exemplified using a B6D2F1 clone (line C4) shown at 12 weeks with her litter. FIG. 2D depicts the PCR analysis of microsatellite markers in genomic DNA from ntES cell lines (CN1, CN2, CN3, CN4) and cloned offspring (cCN1) confirms the clonal origin of the C57BL/6^{nu/nu} pup derived from line CN1. Polymorphic markers D8Mit248, D9Mit191 and D4Mit204 are conserved between genomic DNA from the ntES cell lines and the cloned pup, but differ from those of the ICR surrogate mother (CD1) or ooplast recipient strain, B6D2F1 (F1).

FIGS. 3A-3D show the characterization of nuclear transfer ES (ntES) cells in vitro. FIG. 3A shows phase contrast microscopy of representative ntES cells at passage five. FIG. 3B shows that ntES cells readily formed embryoid bodies. FIG. 3C depicts that staining of near-confluent cultures for the undifferentiated ES cell marker, alkaline phosphatase reveals islands of undifferentiated ntES cells in the line, C1. FIG. 3D shows the PCR analysis of microsatellite markers D4Mit204 and D7Mit22 in genomic DNA from selected ntES cell lines (C13, C15, C16, C17) and mouse strains used in their derivation, showing a conserved amplimer profile with that of 129F1 nucleus donor strains D1 and D2, but not those of the oocyte donor (F1) or surrogate mother (CD1).

FIG. 4 shows the multi-lineage differentiative potential of ntES cells. Embryoid bodies derived from ntES cell lines were differentiated for nine days in vitro. Immunohistochemical analysis revealed positive staining for markers characteristic of endodermal lineage (Troma-1 and alpha-fetoprotein), mesodermal lineage (myosin, fibronectin and smooth muscle actin) and ectodermal lineage (nestin, PSA-NCAM and cytokeratin) as indicated. All three lines exhibited totipotent potential, differing in the quantitative distribution of the various markers. Images shown are for C15 and C16. Scale bar=25 μm in all panels.

FIG. 5 shows the five distinct steps for the derivation of dopaminergic neurons from mouse ntES cells.

FIG. 6 shows the expression of specific midbrain transcription and patterning factors by the ntES derived dopamine neurons.

### DETAILED DESCRIPTION OF THE INVENTION

Embryonic stem (ES) cells are fully pluripotent in that they can differentiate into all cell types, including gametes. The present invention provides 35 ES cell lines that have been derived via nuclear transfer (ntES cell lines) from adult mouse somatic cells derived from inbred, hybrid and mutant strains. The ntES cells of the present invention were found to be capable of contributing to an extensive variety of cell types, including dopaminergic and serotonergic neurons in vitro and germ cells in vivo. Furthermore, cloning by transfer of ntES cell nuclei can result in normal development to fertile non-human adults. The present invention therefore provides fully pluripotent ntES cells.

One particular aspect of the present invention provides for the first time a method of generating neuronal cells from nuclear transfer ES cells that synthesize dopamine (dopaminergic neurons) and serotonin (serotonergic neurons). Furthermore, the methodology disclosed herein allows the efficient generation of ntES cells, which heretofore were obtained with very low efficiency. Indeed, the present invention allows the production of unlimited numbers of isogenic dopamine neurons.

The methodology disclosed herein can be readily applied to the generation of human ntES cells, and furthermore is of great clinical relevance for the generation of dopamine neurons for transplantation therapy in Parkinson's disease (PD). Indeed, whereas there has been great interest in developing alternative renewable cell sources for cell transplantation in Parkinson's disease the only current source is human fetal tissue. Furthermore, the present transplantation procedure requires the use of human fetal tissue derived from up to 4 or even 6 fetuses to obtain an acceptable clinical outcome. This use of such large amounts of fetal tissue raises insurmountable ethical and technical challenges. Indeed, an alternative procedure is required for a more widespread use of a treatment that needs to be provided to greater than one million individuals with Parkinson's disease in the United States alone. The ntES derived dopamine neurons of the present invention offer not only an unlimited supply of dopamine cells, but also the immunological advantage of having cells with the same genetic make-up as the patient. Such cells would be completely immunocompatible and therefore would obviate the use of immunosuppressive therapy in grafted patients.

In addition, the ntES cells of the present invention can be used to generate alternative CNS cell types. Such CNS cell types include GABA neurons, oligodendrocytes in Huntington's disease (HD), stroke, epilepsy, and demyelinating disorders. These cell types derived from the ntES cells of the present invention are also part of the present invention. In one particular embodiment the present invention provides a ntES derived oligodendrocyte for brain repair following radiation-induced damage of white matter tracts.

The present invention further provides individualized in vitro assay systems which employ the isogenic cell populations of the present invention (e.g., the neural cells exemplified below). Such in vitro assays can be used for drug testing, for example, or gene discovery. Thus the isogenic cell populations prepared from an individual's own DNA could be utilized as an individualized in vitro system for drug testing or gene discovery, to determine individual susceptibilities to particular carcinogenic factors, and/or other environmental factors. Furthermore, these in vitro assay systems can be used to help predict the effectiveness and/or desirability of alternative treatments, such as anti cancer therapies.

For clinical application ntES derived cells need to be of very high purity to prevent the generation of unwanted tissue types after transplantation. Positive selection using FACS sorting cells tagged with a brain cell specific antibody can therefore be applied. In addition, positive selection can be achieved by introduction of an antibiotic resistance capability that is controlled by a brain stem cell specific promoter. This will allow the selective growth of brain stem cells in medium containing antibiotics and death of non-brain cells which cannot switch on the brain stem specific promoter. Finally negative selection can be achieved via a suicide gene (herpes thyrnidine kinase) driven by an ES cell specific promoter. Upon addition of ganciclovir persisting ES cells in the differentiated culture will thereby be eliminated.

It is also preferred to have appropriate safety checks prior to cell transplantation studies to prevent unwanted mutations in grafted cells. Therefore, in a preferred embodiment, an inducible suicide mechanism could be included in the cells prior to grafting to eliminate the grafted cells in case of any unexpected problem. Thus, remaining undifferentiated ES cells could be eliminated by introducing a construct expressing HSV thymidine kinase under the control of a ES cell specific promoter. Upon differentiation the remaining undifferentiated ES cells could be killed by adding gancyclovir which selectively affects cells that express HSV thymidine kinase. Other suicide mechanisms can be used in a similar fashion.

The present invention can also be used for the rescue and propagation of sterile mouse phenotypes. For example, a sterile mouse (e.g., azoospermia) could be rescued either by germ line transmission in the context of a non-sterile chimera, or following nuclear transfer. Since ES cells support recombination at a relatively high efficiency, known mutations in ntES cells might be repaired by gene targeting or transfection before they are used to establish germ line chimeras or in cloning. This facilitates the establishment of germ cells and individuals containing multiple targeted alleles.

In addition, the methodology provided herein can be used to treat mitochondrial defects in laboratory animals. Laboratory animals such as mice or cells therefrom that exhibit a mitochondrial defect can be rescued by nuclear transplantation into oocytes from a donor with intact mitochondria. This would allow the study of a specific genotype in the context of normal mitochondrial function. This application could be particularly relevant both experimentally and eventually clinically since there are cases, though admittedly rare, of such mitochondrial diseases in humans.

ABBREVIATIONS

| | |
|---|---|
| DMSO | Dimethyl Sulfoxide |
| PSA - NCAM | polysialylated neural cell adhesion molecule |
| B27 | Medium supplement first described by Brewer |
| | et al., [J. Neurosci. Res. 35, 567-576 (1993) |
| EDTA | Ethylenedinitrilo tetraacetic acid |
| bFGF | basic fibroblast growth factor = fibroblast growth factor 2 |
| FGF8b | fibroblast growth factor 8b |
| EGF | epidermal growth factor |
| CNTF | ciliary neurotrophic factor |
| PDGF | platelet derived growth factor |
| T3 | Triiodothyronine, a thyroid hormone |
| BMP | bone morphogenetic protein |
| BDNF | brain-derived neurotrophic factor |
| NT4 | Neurotrophin 4 = Neurotrophihn5 = Neurotrophin4/5 |
| GDNF | Glial cell line derived neurotrophic factor |
| DbcAMP | dibutyryl cyclic adenosine monophosphate |

Therefore, if appearing herein, the following terms shall have the definitions set out below.

As used herein a “nuclear transfer stem cell” or “ntES” is a pluripotent cell that is obtained after the insertion of a nucleus from a cell into an enucleated oocyte.

As used herein the “Inner Cell Mass” or “ICM” of a blastocyst contains all of the progenitor cells that will build the embryonic tissues. The ICM can be located easily using standard microsurgical techniques [see Matise et al., in Gene Targeting: A Practical Approach A. L. Joyner Ed. (Oxford University Press), pp. 129-131 (2000)].

As used herein the term “container” is used to indicate a solid substrate or support” that provides surface for a cell to grow and/or differentiate and/or allows for a volume of liquid to cover or contain the cell. Preferably the containers are made from glass or a plastic. Particular examples of solid supports used herein are laboratory flasks, petri dishes and glass slides, i.e., the types of containers used in standard tissue culture procedures.

As used herein a “cumulus cell” is a cell of the inner mass of granulosa cells surrounding the oocyte.

As used herein “embryoid bodies” or (EBs) are aggregates of differentiating ES cells that mimic in vitro the events of gastrulation occurring in the embryo in vivo. EBs contain cells of all three lineages: ectoderm, endoderm and mesoderm.

The present invention provides methods for converting ntES cells to fully differentiated cells in vitro. In a particular embodiment exemplified below, ntES cells are fully differentiated to produce neurons, and more particularly dopaminergic neurons.

Initially, a somatic cell can be obtained from any mammalian subject. Suitable mammalian subjects include humans and any other non-human animal mammal such as rodents, e.g., mice, rats, rabbits, and guinea pigs; farm animals e.g., sheep, goats, pigs, horses and cows; domestic pets such as cats and dogs, higher primates such as monkeys, and the great apes such baboons, chimpanzees and gorillas.

As exemplified below, a somatic cell is obtained from the tail of a mouse or alternatively from the cumulus oophorus. In the Examples below, cumulus cells were acutely isolated immediately prior to nuclear transfer as described previously [Wakayama et al., Nature 394, 369 (1998)] whereas the tail tip nucleus donors were from 5-7 day-old primary cultures [Wakayama and Yanagimachi, Nat. Genet. 22, 127 (1999)]. The nucleus of the somatic cell can then be microinjected (preferably by piezo electrically-actuated microinjection) into an enucleated oocyte.

Each resulting embryo is placed into an individual compartment, a well of a 96-well plate was used in Example 1 below, and then seeded with embryonic fibroblast feeders. After a reasonable time (e.g., two days to two weeks) colonies of undifferentiated cells are detached from the compartment and transferred to a new compartment that contains fresh medium and is seeded with fresh embryonic fibroblast feeders.

Clonal expansion of undifferentiated ntES cells is then carried out in the absence of feeder cell layers over a one to two day period. The resulting ntES cells are then isolated and cultured. The cells are then split 1:3 or 1:4 every one to two days. Cells at this stage show all the typical characteristics of “normal” ES cells such as growth pattern, alkaline phosphatase reactivity, embryoid body formation and others. These embryoid bodies are then ready for treatment as described in the Examples below, to generate any desired differentiated cell.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLES

### Differentiation of Embryonic Stem Cell Lines Generared From Adult Somatic Cells by Nuclear Transfer

### Results

Stem cells are able to differentiate into multiple cell types, representatives of which might be harnessed for tissue repair in degenerative disorders such as diabetes and Parkinson's disease [McKay, Nature 406, 361 (2000)]. One obstacle to therapeutic applications is obtaining stem cells for a given patient. A solution would be to derive stem cells from embryos generated by cloning from the nuclei of the individual's somatic cells. Previously, mice have been cloned by microinjection using a variety of cell types as nucleus donors, including embryonic stem (ES) cells [Wakayama et al., Nature 394, 369 (1998), Wakayama and Yanagimachi, Nat. Genet. 22, 127 (1999), Wakayama et al., Proc. Natl. Acad. Sci. USA 96, 14984 (1999)]. However, heretofore, the converse experiment had not been performed, i.e., deriving ES cell lines in vitro from the inner cell mass (ICM) of blastocysts clonally produced by nuclear transfer.

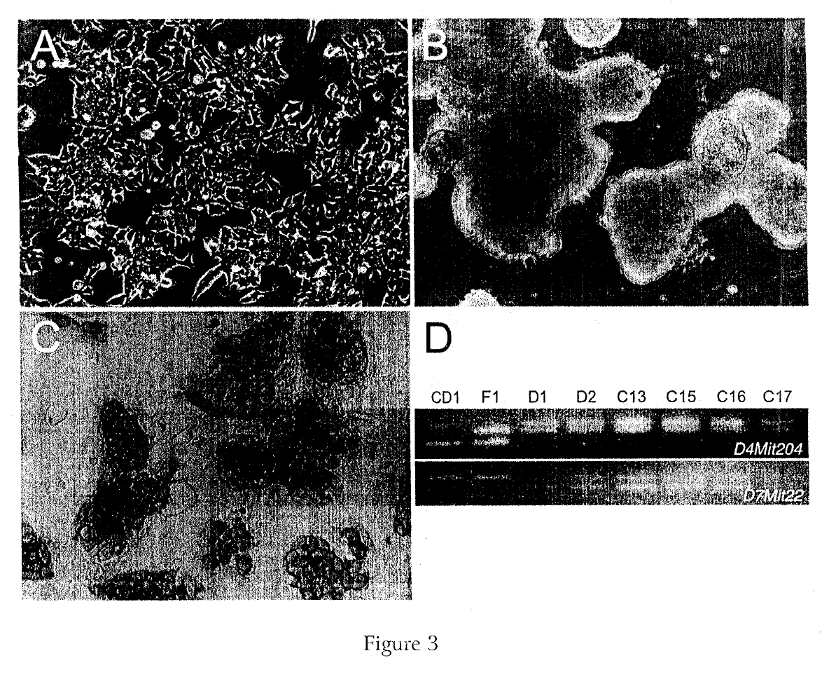

To this end, nuclei from adult-derived somatic donor cells of five mouse strains, including inbred (eg 129/Sv and C57BL/6^{nu/nu}, nude) and F1 hybrid (e.g., C57BL/16×DBA/2) representatives were transferred by microinjection (see methods below) to produce cloned blastocysts (Table 1). When plated on fibroblast feeder layers in culture medium (see methods below) cloned blastocysts from all five strains tested yielded at least one nuclear transfer ES (ntES) cell line (Table 1, FIGS. 3A-3D). Cultures were established from XX embryos derived via cumulus cell nuclear transfer (14.2% of blastocysts) and both XX and XY embryos derived from tail-tip cells (6.5%; Table 1). In total, 35 successfully cryopreserved stable ntES cell lines were produced.

TABLE 1
Establishment of ntES cell lines following nuclear transfer from adult-derived cumulus or tail tip cells and examination of
pluripotency following injection into fertilization-derived blastocysts.

| | | | Establishment of ntES cell vial nuclear | | | In vivo differentiation after ntES cell injection into blastocysts^{†} | | | |
|---|---|---|---|---|---|---|---|---|---|
| | | | transfer | | | | | | No. germ line |
| | | | No. | Blastocyst | Established | | No. | No. | transmitting cell |
| Nucleus donor | | | reconstructed | development | ntES cell | % normal | injected | chimeras/ | lines |
| Strain | Sex | Tissue | oocytes | (%) | line (%)* | karyotype^{‡} | blastocysts | offspring | (black/pups)^{§} |
| B6D2F1 | F | Cumulus | 130 | 57 (43.8) | 9 (15.8) | 67.8 ± 14.1 | 129 | 39/102 | 1 (5/196) |
| | | | | | [6.9] | (6) | | | |
| 129/Sv | F | Cumulus | 44 | 13 (29.5) | 1 (10.0) | 51.8 (1) | 25 | 2/15 | 1 (1/72) |
| | | | | | [2.3] | | | | |
| 129/Sv | M | Tail tip | 88 | 42 (47.7) | 1 (2.4) [1.1] | 66.2 (1) | 24 | 17/20 | 1 (2/127) |
| 129F1 | M | Tail tip | 182 | 54 (29.7) | 7 (13.0) | 50.5 ± 16.7 | 49 | 16/25 | 1 (3/100) |
| | | | | | [3.8] | (4) | | | |
| C57BL/6^{nu/nu} | F | Tail tip | 159 | 75 (47.2) | 5 (6.7) [3.1] | 25.8, 31.3 (2) | 24 | 4/22 | 0 |
| C57BL/6^{nu/nu} | M | Tail tip | 210 | 88 (41.9) | 4 (4.5) [1.9] | 46.1 ± 33.0 | 44 | 16/25 | 2 (10/119) |
| | | | | | | (3) | | | |
| EGFP Tg | F | Cumulus | 118 | 50 (42.4) | 7 (14.0) | 10.3 (1) | 14 | 3/13 | — |
| | | | | | [5.9] | | | | |
| EGFP Tg | M | Tail tip | 85 | 19 (22.4) | 1 (5.3) [1.2] | 68.8 (1) | 39 | 8/15 | 1 (8/31) |
| Total (%) [%] | | | 1016 | 398 (39.2) | 35 (8.8) | 48.8 ± 20.4 | 355 | 105/237 | 7 (24/645) |
| | | | | | [3.4] | (19) | | | |

*Expressed as % of blastocysts ( ) and of reconstructed oocytes [ ]
^{†}Data refer to karyotyped ntES cell lines only.
^{‡}More than 50 M-phase cells were examined for each ntES cell line. Number of ntES cell lines examined is shown in parentheses.
^{§}Data are shown for ntES cell lines that exhibited germ line transmission in chimeras. Data from non-transmitting chimeras has been omitted.

Clonal origin of ntEs cell lines was confirmed by PCR analysis of polymorphic markers (FIGS. 3A-3D, see methods below). The ntES cell morphology of most lines was similar to that of widely disseminated lines such as E14 [Hooper et al., Nature 326, 292 (1987)].

No evidence was found for a pronounced difference in the efficiency of ntES cell line establishment between inbred and hybrid backgrounds (Table 1). All ntES cell lines tested expressed markers diagnostic for undifferentiated ES cells (see methods below) including alkaline phosphatase (FIGS. 3A-3D) and Oct3/4.

ES cells have been induced to differentiate in vitro to produce cardiomyocytes [Metzger et al., J. Cell Biol. 126, 701 (1994)], neurons [Lee, et al., Nature Biotechnol. 18,675 (2000)], astrocytes and oligodendrocytes [Brustle et al., Science 285, 754 (1999)] and hematopoietic lineages [Kennedy et al., Nature 386, 488 (1997)]. In order to assess the pluripotency of ntES cells, it was first sought to differentiate them in vitro to a wide variety of ectodermal, mesodermal and endodermal lineages, and second to induce a highly differentiated cell type. A particularly specialized example was chosen with therapeutic potential: dopaminergic neurons.

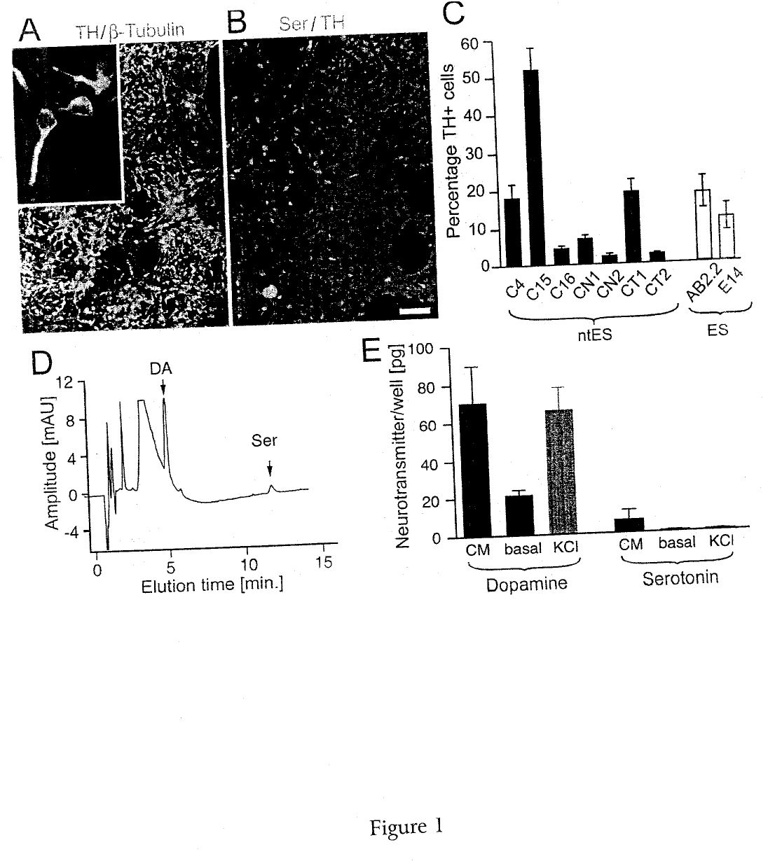

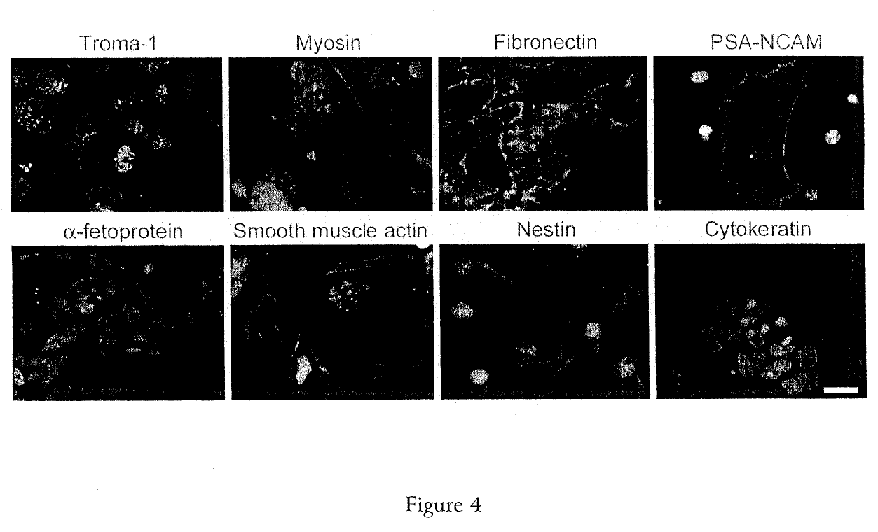

Differentiation of embryoid bodies (FIGS. 3A-3D, see methods below) derived from three different ntES cell lines resulted in a mixed population of ectodermal, endodermal and mesodermal derivatives (FIG. 4). Efficient neural differentiation of ntES cells could be readily induced in each of the seven lines tested. Generation of specific midbrain dopaminergic neurons from ntES cells was achieved with a range of efficiencies using a multistep differentiation protocol described previously [Lee et al., Nature Biotechnol. 18, 675 (2000), see methods below] (FIG. 1). One ntES cell line yielded dopaminergic neurons in excess of 50% of the total cell number. The functional nature of these neurons was confirmed by reverse phase HPLC (RP-HPLC) determination of dopamine release (see methods below). Serotonergic neurons were also detected histochemically, though in smaller numbers, and serotonin release was confirmed by RP-HPLC (FIGS. 1D, 1E).

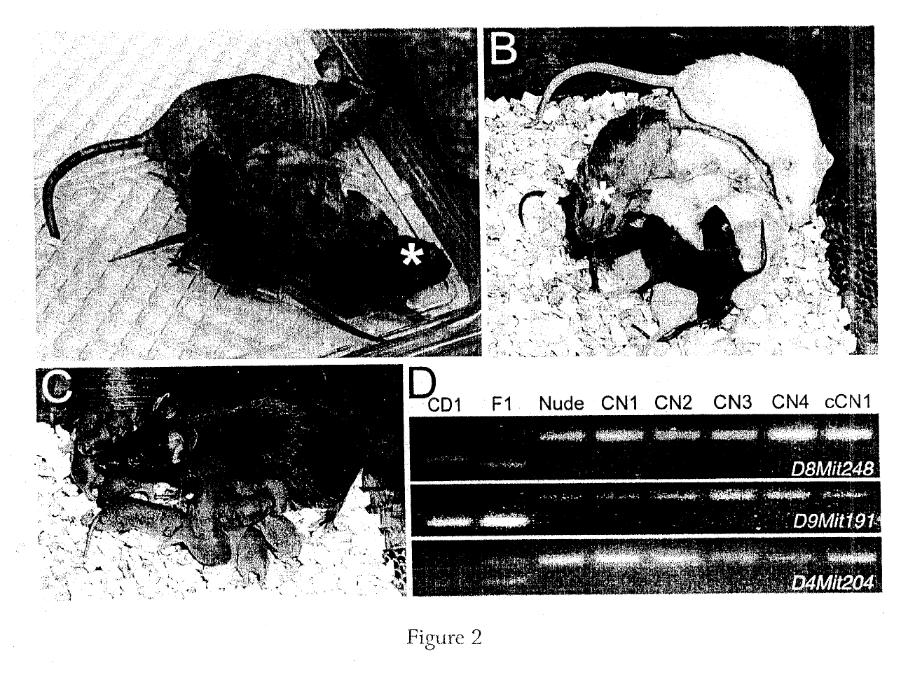

Two recent reports [Munsie et al., Curr. Biol. 10, 989 (2000), Kawase et al., Genesis 28, 156 (2000)] describe a total of five mouse ES cell-like lines derived from the ICMs of cloned blastocysts, although none contributed to the germ line. The contribution of 19 ntES cell lines to chimeric offspring were characterized following their injection into fertilization-derived ICR blastocysts (see methods below). Table 1 summarizes the contribution of ntES cells to 105 chimeric offspring following 355 blastocyst injections. The contribution can be readily approximated by coat color since all ntES cell lines are derived from black-eyed strains with dark coat color, whereas the ICR mouse is an albino mouse (FIGS. 2A-2B). ntES cell lines generally contributed strongly to the coats of chimeric offspring (Table 1). This was corroborated for ntES cells derived from a hybrid strain ubiquitously expressing high levels of the reporter transgene, EGFP. [The line EGFP Tg contains a transgene expressing enhanced green fluorescent protein (EGFP) under the control of a CMV-IE enhancer/chicken Beta-actin promoter combination active in most, if not all, tissues.] All internal organs examined from two EGFP Tg chimeras contained an extensive contribution from the EGFP-expressing ntES cells.

As a comprehensive measure of pluripotency, the ability to contribute to the germ line is considered a defining characteristic of ES cells. Chimeric offspring were crossed with the albino mouse strain, ICR. In ongoing experiments, 24 pups have been derived following chimera×ICR crosses as judged by eye and coat color and where appropriate, EGFP expression (Table 1). Germ line transmission was demonstrated for seven ntES cell lines derived from male and female representatives of all mouse progenitor strains. These data confirm that ntES cells contribute to both male and female gametogenesis when derived from either inbred, hybrid or mutant strains (Table 1), consistent with the universality of the phenomenon among diverse genetic backgrounds.

To determine whether the reprogramming that produced fully pluripotent ntES cells could be reversed, it was attempted to re-derive the original nucleus donor cell types in offspring cloned by nuclear transfer from ntES cells [Wakayama et al., Nature 394, 369 (1998)]. Nuclei from all ntES cell lines supported development in vitro to the blastocyst stage following microinjection into enucleated oocytes (Table 2). When transferred to pseudopregnant surrogate mothers, blastocysts derived from six of the ntES cell lines developed to term, resulting in a total of 20 live-born pups. Of these, one was derived from the nucleus of a C57BL/6^{nu/nu} (nude, inbred) background, and the remaining 19 from the nuclei of hybrid strains (FIG. 2C; Table 2). Hybrid genomes thus preferentially supported cloning in these experiments. Moreover, 11 (all cumulus-derived females; see FIG. 2A) of the 19 were derived from B6D2F1 ntES cell lines, of which 10 survived to adulthood, and are healthy, exhibiting normal fertility. The remaining nine, which died perinatally of unknown cause(s), also contained genomic contribution from the hybrid, B6D2F1(B6D2F1×129/Sv; Table 2), albeit diluted. This possibly reflects a subtle, yet critical contribution made by the hybrid genetic background of B6D2F1. The clonal origin of ntES cells and cloned offspring by PCR analysis of polymorphic markers were corroborated (FIG. 2D).

It was also demonstrated that adult-derived somatic cell nuclei can efficiently be used to generate ES cell lines that exhibit full pluripotency; they can be caused to differentiate along prescribed pathways in vitro, contribute to the germ line following injection into blastocysts, and support full development following nuclear transfer. Since ES cells support homologous recombination at a relatively high efficiency, genetic lesions in ntES cells might be repaired by gene targeting or transgenic complementation before they are used to establish germ line chimeras or in cloning. This facilitates the establishment of germ cells, individuals and cell lines containing targeted alleles.

Reports of human ES cell-like cell lines [Thomson et al., Science 282, 1145 (1998), Shamblott et al., Proc. Natl. Acad. Sci. U.S.A. 95, 13726 (1998)] coupled to the success of mammalian cloning by somatic cell nuclear transfer, have raised the possibility that ntES cells could provide a source of differentiated cells for human autologous transplant therapy; therapeutic cloning [Shamblott et al., Proc. Natl. Acad. Sci. U.S.A. 95, 13726 (1998)]. In this context, demonstration of the full pluripotency of ntES cells is particularly relevant; for example, adult-derived stem cells are apparently restricted in their range of potential cell fates and may be unable to contribute to all tissues including hematopoietic lineages [Clarke et al., Science 288, 1660 (2000)]. Indeed, the efficient generation of midbrain dopaminergic neurons in vitro has been achieved to date only with mesencephalic precursors [Studer et al.,Nature Neurosci. 1, 90 (1998)] and ES cells [Lee, et al., Nature Biotechnol. 18, 675 (2000)], but not from adult-derived cells. In combining ES and nuclear transfer technologies, this limitation has been addressed herein and it has been demonstrated that the first steps required for the application of cloning to transplant therapy is feasibile.

TABLE 2
Cloning using ntES cells as nucleus donors.

| | | | Nuclear transfer | | | | |
|---|---|---|---|---|---|---|---|
| Nucleus donor | | | and in vitro development | | In vivo development | | |
| | | No. | No. | Morula/Blastocyst | Implantation sites/ | | Live cloned |
| | | ntES | reconstructed | development | transferred | Fetuses/ | offspring |
| Strain | Sex | cell lines | oocytes | (%) | embryos | placentae | (%) |
| B6D2F1 | F | 6 | 933 | 386 (41.4) | 294/386 | 15 | 11 (2.8) |
| 129 | F | 1 | 181 | 26 (14.4) | 10/26 | 0 | 0 |
| 129 | M | 1 | 296 | 146 (49.3) | 86/146 | 22 | 0 |
| 129F1 | M | 4 | 712 | 199 (27.9) | 166/196 | 24 | 8 (4.0) |
| C57/BL/6^{nu/nu} | M | 2 | 675 | 88 (19.0)* | 82/175 | 2 | 1 (0.6) |
| EGFP Tg | M | 1 | 168 | 46 (27.4) | 25/46 | 1 | 0 |
| Total (%) | | 15 | 2965 | 803 (27.1) | 663/975 | 64 | 20 (2.1) |

*87 embryos were transferred to recipient females at the 2-cell stage to avoid the 2-cell block.

### Methods

Generation of Cloned Blastocysts: The mouse strains used were B6D2F1 (C57BL/6×DBA/2), 129/SvTac, 129F1 (129/SvTac×B6D2F1), nude (C57BL/6^{nu/nu}) and EGFP Tg (B6D2F2×ICR, F6). 8-15-week-olds were used as nucleus donors, with recipient oocytes from 8-10-week-old B6D2F1s. Cumulus cells were acutely isolated immediately prior to nuclear transfer as described previously [Wakayama et al., Nature 394, 369 (1998)]. Tail tip nucleus donors were from 5-7 day-old primary cultures presumed to be fibroblasts [Wakayama and Yanagimachi, Nat. Genet. 22, 127 (1999)]. Cloned embryonic day 3.5 blastocysts were produced by transfer of cumulus or tail tip cell nuclei from 8-12 week old mice [Wakayama et al., Nature 394, 369 (1998)], Wakayama and Yanagimachi, Nat. Genet. 22, 127 (1999)].

Derivation of ntES cells: Cloned embryos were used to establish nuclear transfer ES (ntES) cell lines essentially as outlined previously [Matise et al., in Gene targeting: a practical approach A. L. Joyner Ed. (Oxford University Press), pp. 129-131 (2000)]. Each embryo was placed into one well of a 96-well plate seeded with ICR embryonic fibroblast feeders. After seven days, colonies of undifferentiated cells were detached by trypsinization and transferred to a 96-well plate containing fresh medium and seeded with fresh embryonic fibroblast feeders.

Clonal expansion of undifferentiated ntES cells proceeded after mild trypsinization and sequential transfer to 48-, 24-, 12- and 6-well plates, and finally into a 12.5cm² gelatinized flask (all in the absence of feeder cell layers) at intervals of one to two days. ntES cells were isolated and cultured in “DMEM for ES cells”(Specialty Media, Phillipsburg, N.J.) supplemented with either 15% heat-inactivated fetal calf serum (FCS) (Hyclone) or 15% Knockout Serum Replacement (Life Technologies), and 1000 U leukemia inhibitory factor (LIF)/ml (Gibco) plus the following (Specialty Media): 1% penicillin-streptomycin, 1% L-glutamine, 1% non-essential amino acids, 1% nucleosides, and 1% beta-mercaptoethanol. Cells were split 1:3 or 1:4 every 1-2 days. Routine culture was in the absence of feeder cells. Cells at that stage show all the typical characteristics of “normal” ES cells such as growth pattern, Alkaline phosphatase reactivity, EB formation and others. Confirmation of donor-derived nucleus was carried out by PCR for polymorphic markers (see FIGS. 3A-3D and below). The differentiation potential of ntES cells was further demonstrated by in vitro generation of cells with endodermal, mesodermal and ectodermal identity, (see FIG. 4). In vivo ES properties of ntES cells were demonstrated by chimerism and by germ-line transmission to create a ntES-derived cloned mouse (FIGS. 2A-2D).

PCR analysis: PCR analysis was employed to confirm the genotypes of strains and cell lines. Primer pairs D4Mit204, D7Mit22, D8Mit248 and D9Mit191 [Dietrich et al., Genetics 131, 423 (1992)] (Mappairs, Research Genetics Huntsville, Ala.) corresponding to microsatellite markers were used to generate a profile of PCR amplimers diagnostic for each genotype. 30 microliter reactions containing approx. 50-100 ng genornic DNA from ntES cells or tail tip biopsies were subjected to 34 cycles of PCR (1 min 95° C., 1 min 60° C., 2 min 72° C.) and products separated on a 4% agarose gel (Nusieve 3:1, BMA) prior to visualization.

Staining Procedures: Standard staining procedures were employed throughout. Immunohistochemistry was with the following antibodies: Oct3/4, monoclonal 1:200 (Sigma); TROMA-1, monoclonal, supernatant 1:1 (DSHB, provided by P. Brulet and R. Kemler); myosin, monoclonal 1:200 (Sigma); fibronectin, polyclonal 1:1000 (Sigma); PSA-NCAM (12E3), monoclonal 1:500, (kindly provided by U. Rutishauser and T. Seki); alpha-fetoprotein, polyclonal 1:125 (Chemicon); smooth muscle actin, monoclonal 1:500 (Sigma); nestin (#130), polyclonal 1:1000 (kindly provided by R. McKay); pan-cytokeratin, monoclonal 1:50 (Sigma); β-III tubulin (TUJI), monclonal 1:500 (BabCo); TH, polyclonal 1:250, (Pel Freeze); TH, monoclonal 1:2500 (Sigma); serotonin, polyclonal 1:2000 (Sigma). Cy2- and Cy3- labeled secondary antibodies (Jackson ImmunoResearch) were used for detection as appropriate, and DAPI (Sigma) for nuclear counterstaining.

Pluripotency assay: Culture conditions for pluripotency assay were as follows. ES cells were plated on uncoated bacterial dishes (2×10⁶ cells/10 cm plate) in ES medium for embryoid body (EB) formation as described previously cells [Lee et al., Nature Biotechnol. 18, 675 (2000)]. Differentiation was induced after trypsinization and transfer to 24-well plates in DMEM containing 10% FCS. Cells were fixed after nine days' culture in vitro.

Induction of dopaminergic differentiation: Induction of dopaminergic differentiation in vitro was as described previously [Lee et al., Nature Biotechnol. 18, 675 (2000)] with the following crucial modification. Cells were cultured for longer during stage III (CNS selection stage), ranging from 9-16 days rather than the usual 6 days. Concentrations of bFGF, SHH, FGF8 (R&D Systems) and ascorbic acid (Sigma) were 10 ng/ml, 500 ng/ml, 100 ng/ml and 100 mM respectively.

Detection of dopamine: Reverse phase-HPLC (RP-HPLC) for the detection of dopamine in neuronally conditioned medium was essentially as described previously [Lee, et al., Nature Biotechnol. 18, 675 (2000)]. Samples were collected seven days after differentiation (Stage V), stabilized with orthophosphoric acid and metabisulfite and subsequently extracted by aluminum adsorption. Separation of the injected samples (ESA Autosampler 540) was achieved by isocratic elution in MD-TM mobile phase (ESA) at 0.7 ml/min. The oxidative potential of the analytical cell (ESA Mod. 5011, Coulochem II) was set at +325mV. Identical conditions were applied for serotonin detection. Results were validated by co-elution with dopamine or serotonin standards under varying buffer conditions and detector settings.

Introduction of ntES into blastocysts: ntES cells were introduced into the cavities of E3.5 ICR blastocysts by piezo-actuated microinjection. Since EGFP Tg ntES cells were derived from albinos of a back-crossed EGFP transgenic strain (B6D2F2×ICR, F6), they were injected into blastocysts derived from the agouti cross, B6D2F1×ICR.

### EXAMPLE 2

### Generaltion Of Midbrain Dopamine Neurons

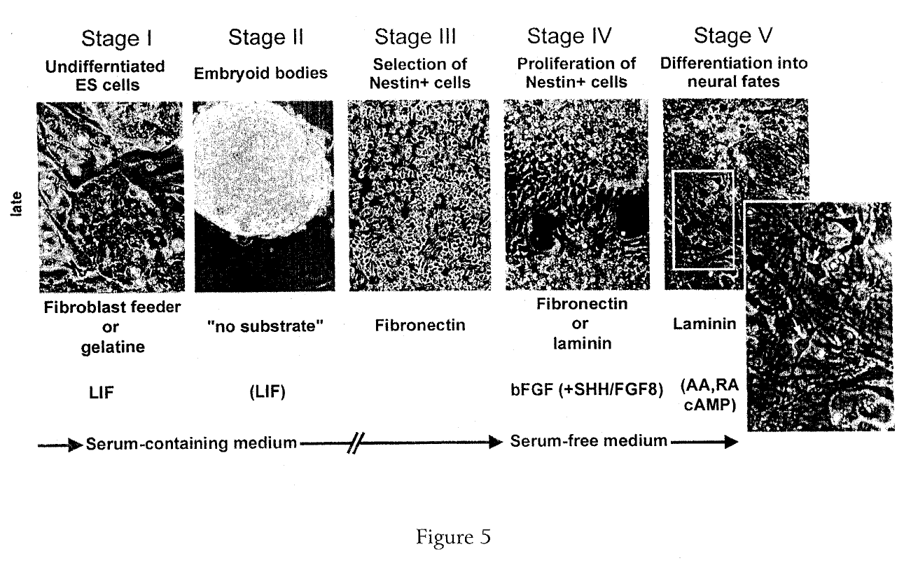

The derivation of dopaminergic neurons from mouse nt ES cells consists of 5 distinct steps, FIG. 5. ntES cells are initially proliferated under standard mouse ES cell conditions such as growth on culture plates precoated with 0.1% gelatin in knock-out DMEM medium (Gibco) supplemented with BME, non-essential amino acids, 15% ES qualified fetal bovine serum and 1000-1500 U/ml LIF(=leukemia) inhibitory factor (ESGRO) as described previously [Lee et al., Nature Biotechnology 18, 675-679 (2000)].

After 3-5 days of ES cell proliferation the stem cells are trypsinized in 0.05% Trypsin/0.02% EDTA for 5 minutes at 37 degrees Celcius. Cell dissociation is stopped by adding serum-containing medium and the cell suspension is spun down in a tissue culture centrifuge at 200 g for 5 minutes. The cell pellet is subsequently resuspended in ES cell medium and cells are plated at about 20-40×10³ cells /cm² on untreated Petri dishes (=Stage II). Over the following 3-6 days free-floating aggregates, so called embryoid bodies (EBs), are being formed. At the end of stage II EBs are collected and spun at low speed (100 g for 3 minutes) and resuspended in ES medium and plated onto culture dishes (Stage II). The following day the medium is changed to a serum free formulation supplemented with fibronectin at 5 μg /ml (ITSFn medium [see, Lee et al., Nature Biotechnology 18, 675-679 (2000)]; containing DMEM/F12+Glucose+Bicarbonate+Insuline, Transferrin, Selenite and Fibronectin).

Unexpectedly, ntES cells require extended growth periods during stage III compared to wild-type ES cells (up to 16 days instead of 5-8 days for wild-type ES cells). After these time periods ntES cell-derived progeny are starting to express the neural stem cell marker nestin and are trypsinzed and replated at 100-200×10³ cells/cm² on polyornithine/laminin coated plates in N2 medium [Studer et al., Nature Neurosci. 1, 290-295 (1998)] supplemented with 10 ng/ml bFGF and 1 μg/ml laminin (Stage IV). To obtain efficient dopaminergic differentiation the following growth factors are required during stage IV: sonic hedgehog (50 ng/ml-1 μg/ml, preferably 500 ng/ml) and FGF8 (10 ng/ml to 250 ng/ml, preferably 100 ng/ml). Stage V is induced by withdrawal of the mitogen bFGF with subsequent differentiation of ES-derived CNS precursors into differentiated neuronal and glial progeny. For the highly efficient generation of dopamine neurons ascorbic acid needs to be added at stage V at a concentration of 20 uM to 500 uM, preferably between 100-200 uM. About 5 days after initiating stage V differentiation large numbers of dopamine neurons are obtained (between 2% to 60% of total cell population) markers (see FIGS. 1A-1E).

Other factors that can further promote dopaminergic differentiation of ntES cells at stage IV and/or V are factors that affect DA neuron induction and survival such as retinoic acid and derivatives, BDNF, NT4, BMP2, BMP4 and/or BMP 7, GDNF, Neurturin, Artemin, dbcAMP, transcription factors such as pax2, pax5 pax8, Nurr1, ptx3, 1mx1b and others.

Modifications of this differentiation protocol allow the efficient generation of other cell types of potential therapeutic interest from ntES cells such as: the generation of astrocytes by replating stage IV cells after trypsinization and subsequent proliferation in bFGF+EGF and bFGF+CNTF (of LIF) followed by factor withdrawal. The generation of oligodendrocytes by replating stage IV cells and proliferating them in the presence of mitogens such as bFGF, EGF and PDGF followed by a period of factor withdrawal. The generation of other neuron subtypes such as GABA neurons for transplantation in Huntington's disease, epilepsy or stroke by growing stage IV cells in the absence of SHH and FGF8 but exposing the cells at stage V to dbcAMP and BDNF or NT4.

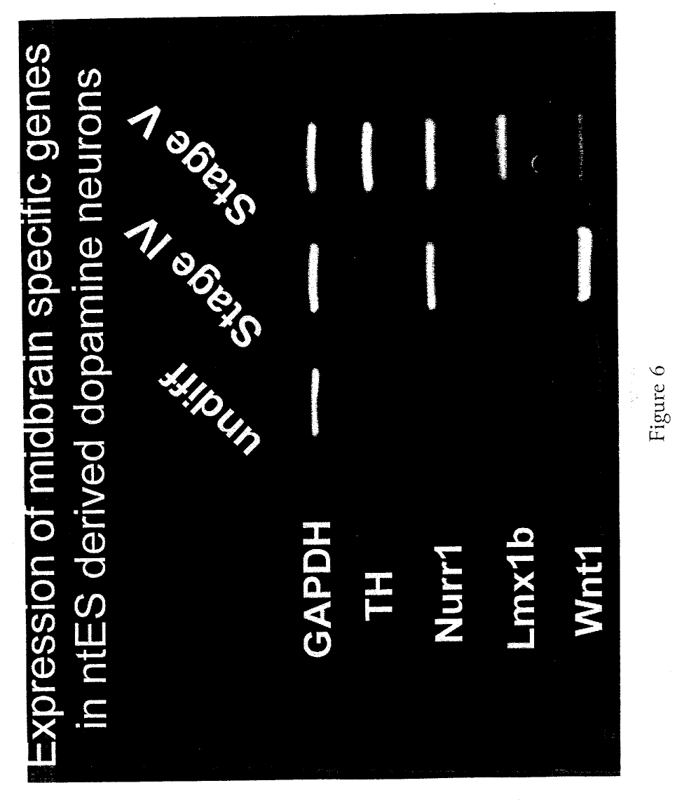

The midbrain identity (as opposed to dopamine neurons in other parts of the brain) of the ntES derived dopamine neurons was confirmed by the expression of specific transcription and patterning factors (see FIG. 6). The function of the dopamine neurons was confirmed by reverse phase HPLC analyses for dopamine and serotonin release as follows: Samples were collected seven days after differentiation (Stage V), stabilized with orthophosphoric acid and metabisulfite and subsequently extracted by aluminum adsorption [Studer,L. et al. Brain Res. Bull. 41, 143-150 (1996)]. Separation of the injected samples (ESA Autosampler 540) was achieved by isocratic elution in MD-TM mobile phase (ESA) at 0.7 ml/min. The oxidative potential of the analytical cell (ESA Mod. 5011, CoulochemII) was set at +325 mV. Identical conditions were applied for serotonin detection. Results were validated by co-elution with dopamine or serotonin standards under varying buffer conditions and detector settings.

### Methods

Seven independent lines of nuclear transfer ES (ntES) cells were differentiated into dopamine neurons. This process has been divided into five distinct stages as depicted in FIG. 5.

Stage I

Undifferentiated ntES cells were grown in T-25 culture flasks in ES medium (described above) supplemented with 1400U/ml leukemia inhibitory factor (LIF), [LIF is sold by Chemicon under the name “ESGRO”, Cat. #ESG 1106], passaged by incubation in 0.05% Trypsin/0.02% EDTA for 10 minutes. The digestion was blocked with FBS-containing ES medium and the cells were spun at 4 degrees C., 1000 rpm (200 g) for 5 minutes. Cells were resuspended in ES medium complemented with 1400 U/ml LIF and cell counts were established. A typical yield of ntES cells ranges from 3-12×10⁶ cells for a T-25 flask.

ES Medium: (per 100 ml)

| | | |
|---|---|---|
| Knock out DMEM medium | 82 ml | Gibco 10829-018 |
| FBS (ES qualified) | 15 ml | Gibco 16141079 |
| MEM (non-essential amino acids 100×) | 1 ml | Gibco 11140050 |
| BME (2-(beta) mercaptoethanol 1000×) | 0.1 ml | Gibco 21985023 |
| PS (penicillin-streptomycin 100×) | 1 ml | |
| L-Glu (L-Glutamine 100×) | 1 ml | Gibco 25030081 |

Stage II

Aliquots of 2.2×10⁶ ntES cells of each line were plated in 7 ml of ES medium +140U/ml LIF on untreated 10 cm petri dishes (Falcon culture plates, catalogue number 1029; these petri dishes are not treated for tissue culture and therefore prevent attachment of EBs). Cells that are not needed for further differentiation studies can be easily frozen at this stage in ES medium+10% DMSO, placed in cryocontainer at −80 degrees C. overnight and maintained for long-term storage in a liquid nitrogen freezer. Medium of EB culture is changed every other day by carefully collecting EBs and a low-speed spin (e.g., 800 rpm for 3 minutes) followed by replacement of the medium. After 4 to 6 days of stage II culture EBs are transferred to stage III conditions as described below. Supplementation with LIF is not absolutely required for stage II cells.

Stage III

Embryoid bodies are collected and spun at low-speed (800 rpm for 3 minutes) followed by a medium change (ES medium with 1400U/ml LIF). The EBs are plated at a ratio of 1:1 (i.e. all EBs obtained from a single dish are placed onto a new dish of the same diameter but of different type). The type of culture plates needed in stage III are tissue culture treated, but uncoated dishes (e.g. Falcon #3003). After 24 hours maintenance of ntES-derived EBs in ES medium +1400U/ml LIF, medium is changed to ITSFn (Insuline, Transferrin, Selenite, Fibronectin medium). It is important to observe the metabolic state of stage III ntES cells at this point in culture because high levels of acid metabolites can be generated leading to pH change of the medium. Such high levels of metabolites can be toxic and an additional medium change or addition of fresh medium might be required. Subsequently, medium changes are carried out every other day. Small phase bright cells will migrate out of the attaching EBs. These cells are the early CNS progenitor population and will start to express CNS markers such as nestin and PSA-NCAM towards the end of stage III. Critically, ntES cells require more extensive time periods in stage III compared to “normal” mouse ES cells (ntES cells ranging from 9 to 16 days, whereas regular ES cells generally require a period of 6 to 8 days in vitro for stage III. If low efficiency of CNS formation is observed medium supplements such as B27 (purchased from Gibco) may be added to improve yield.

Stage IV

Stage III cells covering approximately 70-100% of the surface of the culture plate are ready for progression to stage IV. Cells are trypsinized for 5 minutes in 0.05% Trypsin/0.02% EDTA. The digestion is blocked with ES medium and the cells are spun at 1000-1500 rpm for 5 minutes in a 4 degree C. centrifuge. The cells are resuspended in N2 medium and cell counts established: Typically 5-40×10⁶ cells can be obtained from a single 10 cm stage III plate. Cells are subsequently plated at a cell density of 100-200×10³ cells/cm² on culture plates precoated with polyornithine (15 ug/ml for 1-12 hours followed by laminin lug/ml for 45 minutes-4 hours). The composition of the medium is crucial for determining the type of CNS cell that will be generated. Typically stage IV medium is supplemented with 1ug/ml laminin and 10 ng/ml bFGF allowing for proliferation of immature CNS cells. In addition, factors such as sonic hedgehog (500 ng/ml) and FGF8b (100 ng/ml) are added to increase the ratio of dopamine and serotonin neurons to be generated in stage V. Many additional factors can be added such as EGF, CNTF (both 10 ng/ml) to promote astroglial differentiation, PDGF, T3 or SHH (10 ng/ml each) to promote oligodendroglial fates. However, for glial differentiation best results are obtained when replating stage IV cells again under the stage IV conditions. In the presence of the additional growth factors described above, this second stage IV phase precedes the subsequent differentiation in stage V.

Other factors such as the addition of BMP protein (BMP2, 4 or 7) at stage IV will inhibit dopamine and serotonin neuron generation. Correct cell density at the initial plating stage of stage IV is crucial to allow for good cell survival and total cell yield. Cells are typically grown (proliferated) in stage IV for 6-9 days.

Stage V

Stage V cells are obtained by withdrawal of the mitogenic factors after a medium change. Alternatively, cells can be detached from the plate using a long-term (e.g., an hour) incubation in Ca/Mg free HBSS buffer solution followed by mechanical removal of the cells via pipetman or after careful use of a cell lifter (e.g.; Costar). The cells are subsequently spun at 1000 rpm for 5 minutes and resuspended in N2 medium, the cell number established and cells are plated at 100-200×10³ cell/cm² on precoated culture plates (e.g. costar 24 well plates, Falcon culture plates #3000-series, or other appropriate plates). Depending on the application a variety of attachment substrates might be appropriate. For dopamine neuron differentiation, polyornithine followed by laminin can be used (see above). The use of an ECL (Upstate Biotech) matrix (Entactin-Collagen-laminin) coating appears to give the best results. At stage V (differentiation) the medium typically used is N2 medium in the absence of any mitogens such as bFGF or EGF, but in the presence of ascorbic acid (preferably 50-500 uM final concentration). In addition other factors such as BDNF, NT4, GDNF (all 10-100 ng/ml), dbcAMP (1 mM), all-trans retinoic acid (1-10 nM) and/or other factors promoting dopaminergic differentiation and survival may be added. After 4-10 days large numbers of dopamine neurons can be detected by immunohistochemical analysis or by non-invasive biochemical measurements of dopamine release [Studer, Brain Res. Bull. 41, 143-150 (1996)]. All seven ntES lines tested using this protocol yielded significant numbers of dopamine neurons (see above). Cell differentiation can also be achieved using a reaggregation system as described previously for the differentiation of midbrain precursor cells to be used in intrastriatal transplantation in Parkinsonian rodents [Studer et al., Nature Neurosci. 1, 290-295 (1998)].

The present invention is not to be limited in scope by the specific embodiments described herein. Indeed, various modifications of the invention in addition to those described herein will become apparent to those skilled in the art from the foregoing description and the accompanying Figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

What is claimed is:

1. A method of generating a neuronal cell comprising:
(a) culturing a nuclear transfer embryonic stem (ntES) cell in a first container; wherein an embryoid body (EB) is formed;
(b) removing the EB from the first container, resuspending it in ES cell placing it into a second container;
(c) removing the ES cell medium from the second container and replacing it with serum free media supplemented with fibronectin;
(d) allowing the EB to grow for 9 or more days; wherein the EB expresses the neural stem cell marker nectin;
(e) removing the EB expressing nectin from second container and placing it in a third container coated with polyomithine/laminin; wherein the medium is supplemented with a mitogen, laminin, sonic hedgehog and FGF8; and
(f) withdrawing the mitogen from the medium; wherein a differentiated neuronal cell is formed.

2. The method of claim 1 wherein the neuronal cell is a dopaminergic or serotonergic neuron and the mitogen is bFGF.

3. The method of claim 2 wherein the neuronal cell is a dopaminergic neuron and ascorbic acid is added to step (e).

4. The method of claim 3 wherein one or more of the following factors are added to step (d) and/or step (e): retinoic acid, BDNF, NT4, BMP2, BMP4, and/or BMP7, GDNF, neurturin, artemin, dbbcAMP, pax2, pax5, pax8, Nurr1, ptx3, and 1mx 1b.

5. The method of claim 1 wherein the neuronal cell is an astrocyte, wherein following step (e) but prior to step (f) the EB is removed from the third container and then proliferated on a fourth container with a mitogen selected from the group consisting of bFGF, EGF, and PDGF.

6. The method of claim 1 wherein the neuronal cell is an oligodendrocyte, and wherein following step (e) the EB is removed from the third container and then proliferated in a fourth container with bFGF plus EGF and bFGF plus CNTF (of LIF); and wherein step (f) is performed in medium in which bFGF plus EGF and bFGF plus CNTF (of LIF) are withdrawn.

7. The method of claim 1 wherein the neuronal cell is a GABA neuron and wherein step (e) is performed in the absence of sonic hedgehog and FGF8; and wherein Step (f) is performed in the presence of dbcAMP and BDNF or NT4.

8. A neuronal cell produced from an ntES cell.

9. The neuronal cell of claim 8 that is produced ex vivo.

10. The neuronal cell of claim 9 selected from the group consisting of a dopaminergic neuron, serotonergic neuron, an astrocyte, a GABA neuron, and an oligodendrocyte.
